(19) United States
(12) Patent Application Publication
(10) Pub. No.: US 2020/0323451 A1
(43) Pub. Date: Oct. 15, 2020

(54) Biological Information Measuring Device

(71) Applicant: Seiko Epson Corporation, Tokyo (JP)

(72) Inventor: Katsutoshi MATSUURA, Chino (JP)

(21) Appl. No.: 16/843,924

(22) Filed: Apr. 9, 2020

(30) Foreign Application Priority Data
Apr. 10, 2019 (JP) 2019-074633

(51) Int. Cl. A61B 5/024

(52) U.S. Cl. CPC A61B 5/02427; A61B 5/02438; A61B 2562/0233

(57) ABSTRACT

A biological information measuring device includes: a light-emitting unit emitting irradiation light irradiating an arm; a light-receiving unit receiving reflected light of the irradiation light reflected off the arm; a passage part where the irradiation light and the reflected light pass; and a back lid having a contact surface which surrounds the passage part and comes into contact with the arm, the back lid supporting the passage part. The passage part has an outer surface part coming into contact with the arm, and an inner surface part in a front-back relationship with the outer surface part. The outer surface part has a convex surface protruding from the contact surface and along a first direction from the light-emitting unit toward the passage part and coming into contact with the arm. The inner surface part has a recess part having a bottom surface provided between the contact surface and the convex surface as viewed along a cross section taken from a second direction orthogonal to the first direction.

The present application is based on, and claims priority from JP Application Serial Number 2019-074633, filed Apr. 10, 2019, the disclosure of which is hereby incorporated by reference herein in its entirety.

### BACKGROUND

### 1. Technical Field

The present disclosure relates to a biological information measuring device.

### 2. Related Art

According to the related art, a biological information measuring device for measuring a pulse, which is a kind of biological information, is known. JP-A-2016-47105 discloses this biological information measuring device. According to JP-A-2016-47105, the biological information measuring device has a sensor unit, a body motion noise reduction unit, and a pulsation information computing unit. The sensor unit has a light-emitting unit, a light-receiving unit, and a light-transmitting unit. The light-emitting unit has an LED (light-emitting diode). The light-receiving unit has a photodiode. The light-emitting unit emits irradiation light to a living body via the light-transmitting unit. The light-receiving unit converts reflected light incident thereon via the light-transmitting unit into a detection signal, which is an electrical signal. The body motion noise reduction unit eliminates a noise from the detection signal outputted from the light-receiving unit. The pulsation information computing unit calculates the pulse of a subject, using the detection signal.

A blood vessel through which blood flows is arranged in the living body. The pulsation of the blood vessel is linked to the movement of the heart. The blood vessel absorbs a part of the light emitted from the light-emitting unit. Therefore, the light-receiving unit receives reflected light reflecting the pulsation of the blood vessel. That is, the intensity of the reflected light received by the light-receiving unit changes with time, reflecting the pulsation of the blood vessel. Thus, a pulse wave signal reflects the pulsation of the blood vessel.

The irradiation light emitted from the light-emitting unit passes through the light-transmitting unit and irradiates the living body. A part of the reflected light reflected off the living body passes through the light-transmitting unit and irradiates the light-receiving unit. The light-receiving unit receives the reflected light radiated thereon. The irradiation light emitted from the light-emitting unit spreads as it travels forward. Therefore, the intensity of the irradiation light irradiating the living body is higher when the distance between the light-emitting unit and the living body is shorter. Also, the reflected light reflected off the living body spreads as it travels forward. Therefore, the intensity of the reflected light received by the light-receiving unit is higher when the distance between the living body and the light-receiving unit is shorter.

The intensity of the reflected light received by the light-receiving unit is higher when the distance between the light-emitting unit and the living body and the distance between the light-receiving unit and the living body are shorter. As the intensity of the reflected light received by the light-receiving unit is higher, the ratio of the pulse wave signal to the noise is higher.

Masamichi Nogawa, et al., Medical and Biological Engineering, Vol. 49, No. 6, issued by Japanese Society for Medical and Biological Engineering, December 2011, pages 968-976, is another example of the related art.

In the biological information measuring device disclosed in JP-A-2016-47105, the light-transmitting unit is provided with a recess part. Also, a recess part is arranged at a part of the light-emitting unit, so as to reduce the distance between the light-emitting unit and the living body. However, the intensity of the light received by the light-receiving unit is not high enough to accurately detect the pulse of the living body. A stronger pulse wave signal needs to be detected.

### SUMMARY

A biological information measuring device according to an aspect of the present disclosure includes: a light-emitting unit emitting irradiation light irradiating a living body; a light-receiving unit receiving reflected light of the irradiation light reflected off the living body; a passage part where the irradiation light and the reflected light pass; and a back lid having a contact surface which surrounds the passage part and comes into contact with the living body, the back lid supporting the passage part. The passage part has an outer surface part coming into contact with the living body, and an inner surface part in a front-back relationship with the outer surface part. The outer surface part has a convex surface protruding from the contact surface and along a first direction from the light-emitting unit toward the passage part and coming into contact with the living body. The inner surface part has a recess part having a bottom surface provided between the contact surface and the convex surface as viewed along a cross section taken from a second direction orthogonal to the first direction.

In the biological information measuring device, at least a part of the light-emitting unit may protrude from the contact surface and along the first direction.

In the biological information measuring device, an apex of the convex surface may coincide with a middle point of a straight line connecting a center of the light-emitting unit and a center of the light-receiving unit, as viewed in a plan view taken from the first direction.

In the biological information measuring device, the bottom surface may have a lens.

In the biological information measuring device, the back lid may have a light-shielding part where light does not pass. The light-shielding part may be provided between the contact surface and an apex of the convex surface, as viewed along a cross section taken from the second direction.

### BRIEF DESCRIPTION OF THE DRAWINGS

FIG. 1 is a schematic perspective view showing the configuration of a biological information measuring device according to a first embodiment.

FIG. 2 is a schematic perspective view for explaining the mounted state of the biological information measuring device.

FIG. 3 is a schematic plan view showing the structure of the biological information measuring device.

FIG. 4 is a schematic side cross-sectional view showing the structure of the biological information measuring device.

FIG. 5 is a schematic side cross-sectional view of essential parts for explaining the positional relationship between a light-emitting unit and a light-receiving unit.

FIG. 6 is a schematic side cross-sectional view showing the structure of the light-receiving unit.

FIG. 7 is a schematic view for explaining a method for detecting pulsation of a blood vessel.

FIG. 8 explains the relationship between intra-extravascular pressure difference and intravascular volume.

FIG. 9 shows change in intravascular volume with time.

FIG. 10 is a block diagram showing the configuration for electrical control in the biological information measuring device.

FIG. 11 is a schematic side cross-sectional view showing the structure of a biological information measuring device according to a second embodiment.

FIG. 12 is a schematic side cross-sectional view showing the structure of a passage part according to a third embodiment.

FIG. 13 is a schematic side cross-sectional view showing the structure of a back lid according to a fourth embodiment.

FIG. 14 is a schematic side cross-sectional view showing the structure of a back lid according to a fifth embodiment.

### DESCRIPTION OF EXEMPLARY EMBODIMENTS

Embodiments will now be described with reference to the drawings. The components in the drawings are illustrated on different scales from one component to another in order to make each component recognizable in each drawing.

### First Embodiment

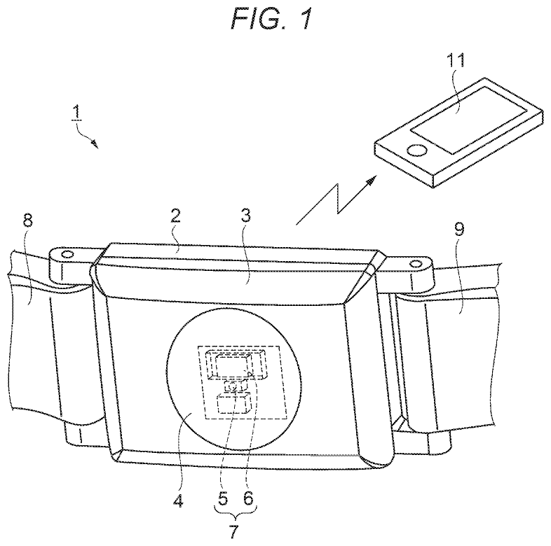

In this embodiment, a characteristic example of a biological information measuring device detecting pulsation of a blood vessel is described with reference to FIGS. 1 to 10. FIG. 1 is a schematic perspective view showing the configuration of the biological information measuring device. As shown in FIG. 1, a biological information measuring device 1 has a box-shaped case 2 having a predetermined thickness. A back lid 3 is installed on one side in the direction of the thickness of the case 2. A passage part 4 where light can pass is arranged in the back lid 3. Inside the case 2, a sensor unit 7 having a light-emitting unit 5 and a light-receiving unit 6 is arranged. The light-emitting unit 5 emits irradiation light. The light-receiving unit 6 receives reflected light of the irradiation light reflected inside a living body.

At the lateral sides of the case 2, a first band 8 and a second band 9 are arranged in such a way as to hold the base 2 between them. At one end of the first band 8, a coupling part, not illustrated, for coupling the first band 8 and the second band 9 together is arranged.

The biological information measuring device 1 has a wireless communication function. The biological information measuring device 1 transmits measured pulse data via wireless communication to an electronic device such as a smartphone 11. The smartphone 11 displays the pulse data measured by the biological information measuring device 1.

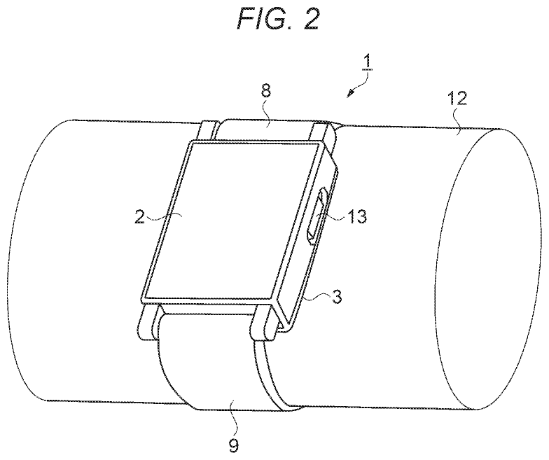

FIG. 2 is a schematic perspective view for explaining the mounted state of the biological information measuring device. As shown in FIG. 2, the biological information measuring device 1 is mounted on an arm 12 of a human body as a living body. The first band 8 and the second band 9 are wrapped around the arm 12 and coupled together at the coupling part. In this way, the biological information measuring device 1 is a wearable device mounted on the arm 12 and measuring biological information of the human body. The biological information measuring device 1 detects a pulse wave signal and computes the number of pulse beats. The pulse wave signal results from observing pressure change or volume change in pulsation of the blood vessel. The number of pulse beats is the number of pulse wave signal peaks per minute.

The biological information measuring device 1 is mounted in such a way that the back lid 3 comes into contact with the arm 12. At this time, the back lid 3 and the passage part 4 come into contact with the arm 12. At a lateral side of the case 2, a USB (universal serial bus) external connector 13 is arranged. The biological information measuring device 1 is charged with electricity via the external connector 13.

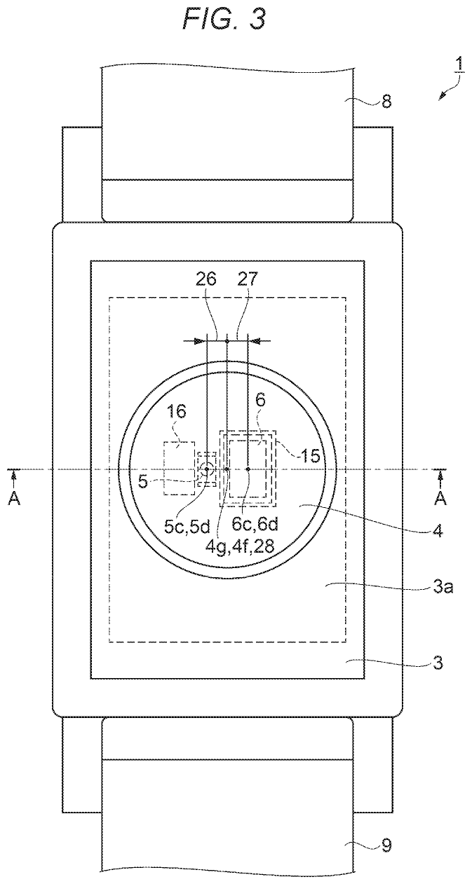

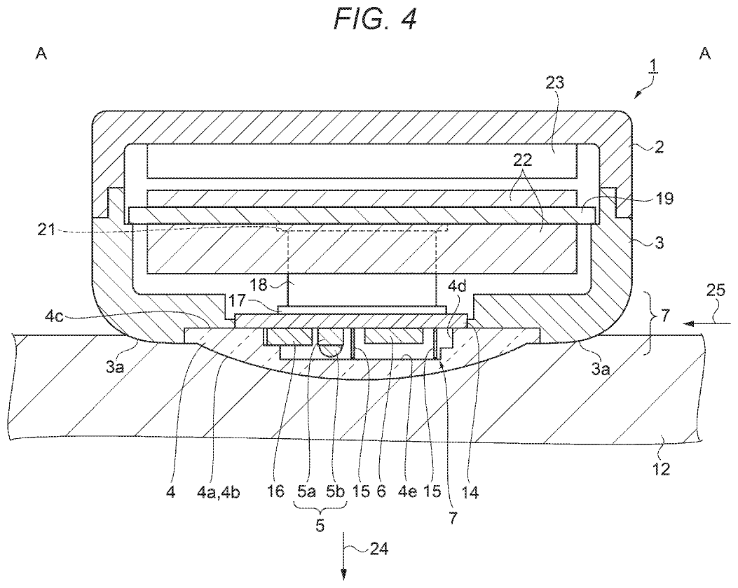

FIG. 3 is a schematic plan view showing the structure of the biological information measuring device. FIG. 3 shows the biological information measuring device 1, as viewed from the side of the back lid 3. FIG. 4 is a schematic side cross-sectional view showing the structure of the biological information measuring device. FIG. 4 shows a cross section taken along A-A in FIG. 3. As shown in FIGS. 3 and 4, the passage part 4 has a circular outer shape and the back lid 3 has a quadrilateral outer shape. The back lid 3 surrounds the passage part 4 and supports the passage part 4.

The sensor unit 7 is arranged relatively near the passage part 4, in a space surrounded by the case 2, the back lid 3, and the passage part 4. The sensor unit 7 has a sensor substrate 14 supported by the back lid 3. The sensor substrate 14 is a rigid substrate. At the sensor substrate 14, the light-emitting unit 5, the light-receiving unit 6, a light-shielding wall 15, and a drive unit 16 are arranged on the side of the passage part 4.

The light-emitting unit 5 emits irradiation light irradiating the arm 12, which is a living body. The light-emitting unit 5 is formed of a light emitter 5a and a lens element 5b. The light emitter 5a is an LED chip formed of a light-emitting element such as an LED (light-emitting diode) sealed with a sealing resin. The light emitter 5a may also be a bare chip formed of a light-emitting element not sealed with a sealing resin. In this embodiment, the light emitted from the light emitter 5a is green light. Green light is reflected off a shallow part of the skin and therefore can irradiate an arteriole. However, the light emitted from the light emitter 5a may be other light than green light.

The lens element 5b condenses the irradiation light to a predetermined depth in the arm 12. The predetermined depth is a depth where an arteriole exists. The material of the lens element 5b is not particularly limited, provided that the material is light-transmissive. For example, an acrylic resin, epoxy resin, glass or the like can be used.

The light-receiving unit 6 receives reflected light of the irradiation light reflected off the arm 12. The light-receiving unit 6 outputs a detection signal representing the amount of the reflected light received. This detection signal is a pulse wave signal. The light-receiving unit 6 is a PD chip formed of a light-receiving element, which is a PD (photodiode), sealed with a sealing resin, though not illustrated in detail. The light-receiving unit 6 may be a bare chip formed of a light-receiving element not sealed with a sealing resin.

The light-receiving element has an n-type semiconductor area on the side of a silicon substrate, and a p-type semiconductor area on the side of a light-receiving surface. When light having sufficiently high energy becomes incident on the p-type semiconductor area, a current is outputted due to a photovoltaic effect. The light-receiving unit 6 is provided with a wavelength-restricting filter which transmits light with the same wavelength as the reflected light but does not transmit other light than the reflected light.

The light-shielding wall 15 is arranged around the light-receiving unit 6. The light-shielding wall 15 is also arranged between the light-emitting unit 5 and the light-receiving unit 6. The light-shielding wall 15 blocks the light traveling from the light-emitting unit 5 directly to the light-receiving unit 6 and thus restrains the irradiation light emitted from the light-emitting unit 5 from becoming incident directly on the light-receiving unit 6 without traveling via the arm 12. The light-shielding wall 15 also restrains stray light, other than the reflected light reflected off the arm 12, from becoming incident on the light-receiving unit 6.

The drive unit 16 is a circuit driving the light-emitting unit 5 and the light-receiving unit 6. The drive unit 16 controls electric power supplied to the light-emitting unit 5. The drive unit 16 also controls the start and stop of the supply of electric power. The drive unit 16 also functions as an AFE (analog front end). The drive unit 16 amplifies an electrical signal outputted from the light-receiving unit 6. The drive unit 16 has a filter. The filter eliminates a noise included in the amplified electrical signal. The drive unit 16 also has an ADC (analog-digital converter). The ADC converts the analog electrical signal into digital data and outputs the digital data.

At a surface of the sensor substrate 14 on the side of the case 2, a first connector 17 is arranged. A flat cable 18 is electrically coupled to the first connector 17. A main substrate 19 is arranged nearer to the case 2 than the sensor substrate 14. At a surface of the main substrate 19 on the side of the sensor substrate 14, a second connector 21 is arranged. The flat cable 18 is electrically coupled to the second connector 21. The flat cable 18 transmits an electrical signal between the first connector 17 and the second connector 21. Also, the first connector 17 and the second connector 21 may be electrically coupled directly together without using the flat cable 18.

At both sides of the main substrate 19, an electrical element 22 such as a CPU, memory, chip resistor, chip capacitor, or antenna is installed. The main substrate 19 takes in a detection signal representing the amount of the reflected light received, inputted from the sensor substrate 14. The main substrate 19 then computes the number of pulse beats. The main substrate 19 transmits data of the number of pulse beats via wireless communication.

A secondary battery 23 is arranged nearer to the case 2 than the main substrate 19. The secondary battery 23 stores electricity supplied from the external connector 13. The secondary battery 23 supplies electric power to the sensor substrate 14 and the main substrate 19. A lithium battery is used as the secondary battery 23.

The passage part 4 is light-transmissive. Therefore, the irradiation light emitted from the light-emitting unit 5 passes through the passage part 4. Also, the reflected light reflected off the arm 12 passes through the passage part 4. The direction from the light-emitting unit 5 toward the passage part 4 is defined as a first direction 24. A part of the passage part 4 that faces into the first direction 24 is defined as an outer surface part 4a. The outer surface part 4a comes into contact with the arm 12. A surface of the back lid 3 that comes into contact with the arm 12 is defined as a contact surface 3a. The contact surface 3a surrounds the passage part 4. The outer surface part 4a has a convex surface 4b protruding from the contact surface 3a and along the first direction 24 and coming into contact with the arm 12.

A part of the passage part 4 that is in a front-back relationship with the outer surface part 4a is defined as an inner surface part 4c. In other words, the passage part 4 has the inner surface part 4c in a front-back relationship with the outer surface part 4a. One direction orthogonal to the first direction 24 is defined as a second direction 25. The second direction 25 is the direction from the light-receiving unit 6 toward the light-emitting unit 5. The inner surface part 4c has a recess part 4d, as viewed along a cross section taken from the second direction 25. The recess part 4d has a bottom surface 4e provided between the contact surface 3a and the convex surface 4b.

An end surface of the sensor substrate 14 that faces into the first direction 24 is in contact with the inner surface part 4c. The light-emitting unit 5, the light-receiving unit 6, the light-shielding wall 15, and the drive unit 16 are accommodated in the recess part 4d. A part of the light-emitting unit 5 protrudes from the contact surface 3a and along the first direction 24. In the first direction 24 from the light-emitting unit 5, the distance between the outer surface part 4a and the bottom surface 4e is short and therefore the distance between the light-emitting unit 5 and the arm 12 is short. This enables the arm 12 to receive intense irradiation light.

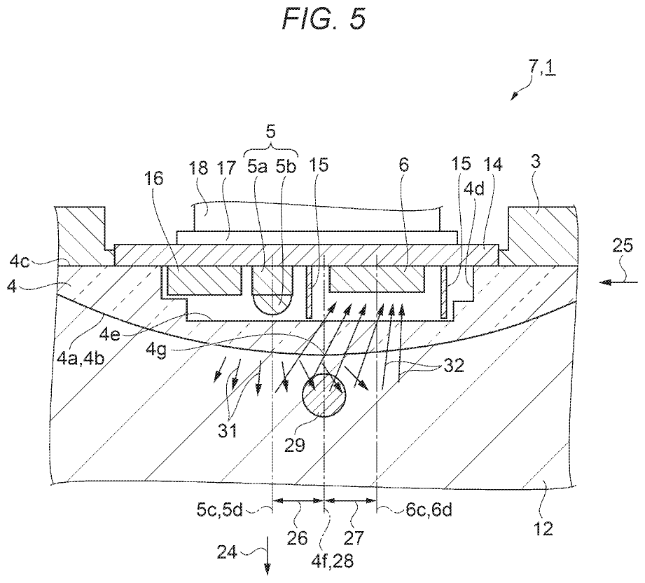

FIG. 5 is a schematic side cross-sectional view of essential parts for explaining the positional relationship between the light-emitting unit and the light-receiving unit. As shown in FIGS. 3 and 5, a centerline passing through a center 5d of the light-emitting unit 5 as viewed in a plan view taken from the first direction 24 is defined as a light-emitting unit center line 5c. A centerline passing through a center 6d of the light-receiving unit 6 as viewed in a plan view taken from the first direction 24 is defined as a light-receiving unit centerline 6c. A centerline passing through an apex 4g of the convex surface 4b as viewed in a plan view taken from the first direction 24 is defined as a convex surface center line 4f. The apex 4g of the convex surface 4b refers to a point protruding most into the first direction 24 on the convex surface 4b.

In this case, the center of the light-emitting unit 5, as viewed in a plan view taken from the first direction 24, is a point where the light-emitting unit center line 5c passes. The center of the light-receiving unit 6, as viewed in a plan view taken from the first direction 24, is a point where light-receiving unit centerline 6c passes. The apex 4g of the convex surface 4b, as viewed in a plan view taken from the first direction 24, is a point where the convex surface center line 4f passes.

The distance between the light-emitting unit center line 5c and the convex surface center line 4f, as viewed in a plan view taken from the first direction 24, is defined as a first distance 26. The distance between the light-receiving unit centerline 6c and the convex surface center line 4f is defined as a second distance 27. In this case, the first distance 26 and the second distance 27 are equal. That is, the apex 4g of the convex surface 4b coincides with the middle point of a straight line connecting the center 5d of the light-emitting unit 5 and the center 6d of the light-receiving unit 6, as viewed in a plan view taken from the first direction 24.

The apex 4g of the convex surface 4b powerfully pressurizes the arm 12. At the pressurized site, the pulsation of the blood vessel changes largely. Therefore, the pulsation of the blood vessel changes largely at the site on the convex surface center line 4f of the arm 12. The site where the pulsation of the blood vessel changes largely passes through the middle point between the light-emitting unit center line 5c in the light-emitting unit 5 and the light-receiving unit centerline 6c in the light-receiving unit 6, when viewed from the first direction 24. A line in the first direction 24 passing through the middle between the light-emitting unit center line 5c in the light-emitting unit 5 and the light-receiving unit centerline 6c in the light-receiving unit 6 is defined as a middle line 28. An inner part of the arm 12 located into the first direction 24 on the middle line 28 is defined as a part to be measured 29.

Irradiation light 31 emitted from the light-emitting unit 5 travels into the arm 12. Then, a part of reflected light 32 reflected off the inside of the arm 12 travels toward the light-receiving unit 6. The distance from the light-emitting unit 5 to the part to be measured 29 and the distance from the part to be measured 29 to the light-receiving unit 6, added together, is defined as a first distance. An arbitrary part other than the part to be measured 29, as viewed in a plan view taken from the first direction 24, is defined as a reference part. The distance from the light-emitting unit 5 to the reference part and the distance from the reference part to the light-receiving unit 6, added together, is defined as a second distance. In this case, the first distance is shorter than the second distance. The light-receiving unit 6 receives light with a higher intensity when the distance the light travels from the light-emitting unit 5 to the light-receiving unit 6 is shorter.

Thus, the part to be measured 29 is a site where the biological information measuring device 1 can measure change in the pulsation of the blood vessel with a high sensitivity. As the apex 4g of the convex surface 4b pressurizes the part to be measured 29, the biological information measuring device 1 can measure a site where the pulsation of the blood vessel changes largely, with a high sensitivity. Even when the convex surface 4b of the biological information measuring device 1 moves along the surface of the arm 12 during exercise or the like, the sensor unit 7 measures the pulsation of the blood vessel at the part to be measured 29 pressed by the convex surface 4b. That is, the biological information measuring device 1 measures a site where the pulsation of the blood vessel changes largely, with a high sensitivity. Therefore, the biological information measuring device 1 can stably measure the pulsation of the blood vessel.

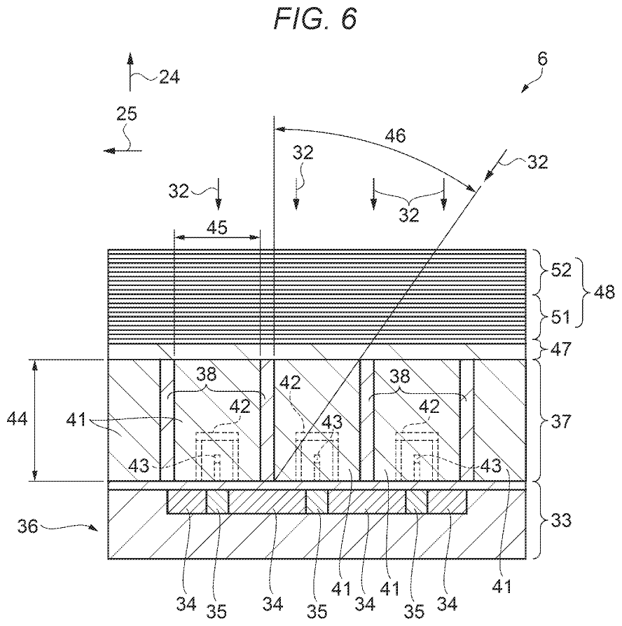

FIG. 6 is a schematic side cross-sectional view showing the structure of the light-receiving unit. As shown in FIG. 6, the light-receiving unit 6 has a silicon substrate 33. The silicon substrate 33 is a P-type substrate. At a part facing into the first direction 24 inside the silicon substrate 33, an N-type diffusion layer and a P-type diffusion layer 35 are alternatively arranged in a planar direction. A p-n junction between the N-type diffusion layer 34 and the silicon substrate 33 forms a photodiode 36. Also, a p-n junction between the N-type diffusion layer 34 and the P-type diffusion layer 35 forms a photodiode. The N-type diffusion layer 34 functions as the cathode of the photodiode. The P-type diffusion layer 35 and the silicon substrate 33 function as the anode.

Further into the first direction 24 from the silicon substrate 33, an angle-restricting filter 37 is arranged. In the angle-restricting filter 37, light-shielding objects 38 are arranged at equal intervals in the second direction 25. The light-shielding object 38 is a film that is thin in the second direction 25. Aluminum, tungsten or the like is used as the material of the light-shielding object 38. A light-transmitting object 41 is arranged between the light-shielding objects 38. For the light-transmitting object 41, any material that can transmit the reflected light 32 with a wavelength received by the photodiode 36 may be used. In this embodiment, for example, silicon dioxide is used as the material of the light-transmitting object 41.

In the angle-restricting filter 37, a first wiring 42 electrically coupled to the N-type diffusion layer 34 is arranged. Also, a second wiring 43 electrically coupled to the P-type diffusion layer 35 is arranged. Tungsten is used for the parts extending in the first direction 24, of the first wiring 42 and the second wiring 43. Aluminum is used for the parts extending in the second direction 25, of the first wiring 42 and the second wiring 43.

The reflected light 32 reaching the light-shielding object 38 is attenuated in light intensity. Therefore, the angle at which the reflected light 32 with a high intensity reaches the photodiode 36 is restricted to within a range of a restricted angle 46. The length of the light-transmitting object 41 in the first direction 24 is defined as a first length 44. The length of the light-transmitting object 41 in the second direction 25 is defined as a second length 45. The restricted angle 46 restricting the reflected light 32 is arctan (second length 45/first length 44). Setting the first length 44 and the second length 45 sets the restricted angle 46. In this embodiment, for example, the first length 44 is 5 μm and the second length 45 is 3 μm. In this case, the restricted angle 46 is 31°.

A protection film 47 is arranged further into the first direction 24 from the angle-restricting filter 37. The same silicon dioxide as the light-transmitting object 41 is used for the material of the protection film 47.

A bandpass filter 48 is arranged further into the first direction 24 from the protection film 47. The bandpass filter 48 is formed of a long-pass filter 51 formed over the protection film 47, and a short-pass filter 52 formed over the long-pass filter 51. The long-pass filter is a filter having the function of passing long-wavelength light and attenuating short-wavelength light. The short-pass filter 52 is a filter having the function of passing short-wavelength light and attenuating long-wavelength light. In this embodiment, for example, the bandpass filter 48 passes light with wavelengths of 500 nm to 600 nm. The long-pass filter 51 and the short-pass filter 52 are thin-film filters formed of thin films staked on each other. The positions in the first direction 24 of the long-pass filter 51 and the short-pass filter 52 may be switched with each other.

A method for manufacturing the light-receiving unit 6 will now be briefly described. First, the photodiode is formed. To form the photodiode 36, the N-type diffusion layer 34 and the P-type diffusion layer 35 are formed at the top of the silicon substrate 33, which is a P-type substrate. The N-type diffusion layer 34 is formed by implanting a group V element such as phosphorus or arsenic into a predetermined pattern in the silicon substrate 33. The P-type diffusion layer 35 is formed by implanting a group III element such as boron into a predetermined pattern in the silicon substrate 33.

Next, the angle-restricting filter 37 is formed. First, in step 1, a silicon dioxide film is deposited by sputtering. In step S2, a hole is formed by photolithography and etching. In step S3, a metal film of aluminum or tungsten is arranged in the hole and over the silicon dioxide film by sputtering. Then, in step 4, the silicon dioxide film is made planar by CMP (chemical-mechanical polishing).

The foregoing steps 1 to 4 are repeated, thus forming the light-shielding object 38 and the light-transmitting object 41. To form the wiring parts in the planar direction of the silicon substrate 33, of the first wiring 42 and the second wiring 43, the metal film formed in step 3 is formed by photolithography and etching. Then, the process shifts to step 1. The angle-restricting filter 37 is formed in this way. The protection film 47 is formed on top of the angle-restricting filter 37. To form the protection film 47, a silicon dioxide film is deposited by sputtering.

Next, the bandpass filter 48 is formed on top of the protection film 47. Anisotropic etching and polishing by CMP are performed on the protection film 47, thus forming an inclined surface of an inclined structure. Next, sputtering of a titanium oxide film and sputtering of a silicon dioxide film are alternately performed, thus forming a multilayer thin film at the inclined surface. The titanium oxide film is a thin film with a high refractive index. The silicon dioxide film is a thin film with a low refractive index. The thickness of the titanium oxide film and the thickness of the silicon dioxide film are adjusted according to the optical properties of the long-pass filter 51 and the short-pass filter 52. This process completes the light-receiving unit 6.

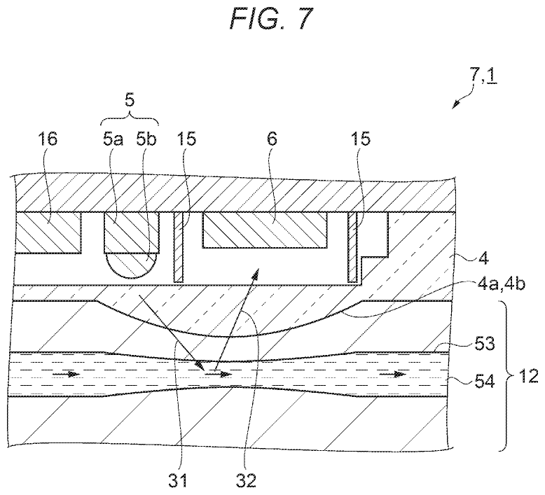

FIG. 7 is a schematic view for explaining a method for detecting the pulsation of a blood vessel. As shown in FIG. 7, an arteriole blood vessel 53 is arranged inside the arm 12. Blood 54 flows inside the blood vessel 53. Due to the cardiac output of the blood 54, the expansion of the blood vessel 53 propagates. The volume of the blood 54 in the blood vessel 53 is defined as an intravascular volume. The intravascular volume is proportional to the cross-sectional area of the section where the blood 54 flows, in the blood vessel 53. The intravascular volume increases when the blood vessel 53 expands. The intravascular volume decreases when the blood vessel 53 contracts. The intravascular volume changes synchronously with the movement of the heart. Since the movement of the heart is linked to the pulsation of the blood vessel, the change in the intravascular volume is linked to the pulsation of the blood vessel.

A part of the irradiation light 31 emitted from the light-emitting unit 5 is absorbed by hemoglobin in the blood 54. A part of the irradiation light 31 that is not absorbed by the hemoglobin is received as reflected light 32 by the light-receiving unit 6. When the intravascular volume increases, the proportion of the irradiation light 31 absorbed by the hemoglobin to the emitted irradiation light 31 increases and therefore the reflected light 32 received by the light-receiving unit 6 decreases. Thus, the light intensity of the reflected light 32 received by the light-receiving unit 6 is linked to the change in the intravascular volume.

Masamichi Nogawa, et al., Medical and Biological Engineering, Vol. 49, No. 6, issued by Japanese Society for Medical and Biological Engineering, December 2011, pages 968-976, discloses information about the relationship between the pressure applied to the blood vessel 53 and the change in the intravascular volume. According to this, applying a pressure similar to the blood pressure to the blood vessel 53 increases the change in the intravascular volume. FIG. 8 explains the relationship between intra-extravascular pressure difference and intravascular volume. In FIG. 8, the horizontal axis represents the intra-extravascular pressure difference. The intra-extravascular pressure difference is the “average pressure inside the blood vessel” minus the “pressure applied to the blood vessel from outside”. The pressure applied to the blood vessel 53 from outside becomes higher toward the left and lower toward the right on the horizontal axis in the illustration. When the convex surface 4b of the passage part 4 is spaced apart from the arm 12, the intra-extravascular pressure difference is in the state on the right side on the horizontal axis in the illustration. When the convex surface 4b of the passage part 4 presses the arm 12, the intra-extravascular pressure difference is in the state close to “0” on the horizontal axis. When the intra-extravascular pressure difference is in the state “0” on the horizontal axis, the average value of the blood pressure in the blood vessel 53 and the pressure applied to the blood vessel 53 by the convex surface 4b of the passage part 4 are equal.

The vertical axis represents the intravascular volume. The intravascular volume becomes larger toward the top and smaller toward the bottom in the illustration. A pressure-volume curve 55 represents the relationship between the intra-extravascular pressure difference and the intravascular volume. The rate of change in the pressure-volume curve 55 indicates the slope of the pressure-volume curve 55. The rate of change in the intravascular volume is high when the slope of the pressure-volume curve 55 is large. The rate of change in the intravascular volume is low when the slope of the pressure-volume curve 55 is small. The rate of change in the intravascular volume is high when the intra-extravascular pressure difference is “0”. The rate of change in the intravascular volume decreases as the intra-extravascular pressure difference goes away from “0”.

The change in the intra-extravascular pressure difference when the contact surface 3a comes into contact with the arm 12 and the convex surface 4b of the passage part 4 presses the arm 12, is defined as a first pressure change 56. The range of the first pressure change 56 represents the intra-extravascular pressure difference that changes due to cardiac output. The first pressure change represents change in the intra-extravascular pressure difference around “0”. The intravascular volume corresponding to the first pressure change 56 is defined as a first volume change 57.

The change in the intra-extravascular pressure difference when the contact surface 3a is spaced apart from the arm 12, is defined as a second pressure change 58. The first pressure change 56 and the second pressure change 58 have the same range of change in pressure difference. In the case of the second pressure change 58, the blood vessel 53 is not pressed by the convex surface 4b of the passage part 4. Therefore, the second pressure change 58 is located more rightward in the illustration than the first pressure change 56. The intravascular volume corresponding to the second pressure change 58 is defined as a second volume change 61.

The slope of the pressure-volume curve 55 at the first pressure change 56 is steeper than the slope of the pressure-volume curve 55 at the second pressure change 58. That is, the rate of change in the pressure-volume curve 55 is higher. Therefore, the range of change of the first volume change 57 is greater than the range of change of the second volume change 61.

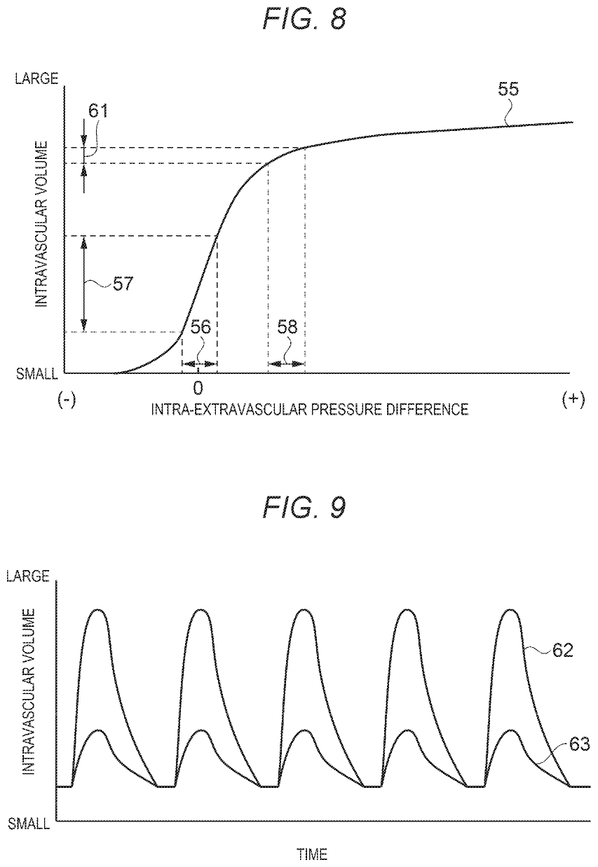

FIG. 9 shows change in intravascular volume with time. The horizontal axis in FIG. 9 represents the lapse of time. Time shifts from left to right in the illustration. The vertical axis represents the intravascular volume. The intravascular volume becomes larger toward the top and smaller toward the bottom in the illustration. A first waveform 62 is a waveform corresponding to the first volume change 57. A second waveform 63 is a waveform corresponding to the second volume change 61. The first waveform 62 and the second waveform 63 are similar shapes. The first waveform 62 has a higher peak of intravascular volume than the second waveform 63. Therefore, as the convex surface 4b of the passage part 4 presses the arm 12 and applies an appropriate pressure to the blood vessel 53, the amplitude of the changing intravascular volume increases. At this time, the sensor unit 7 can more easily detect the pulsation of the blood vessel 53.

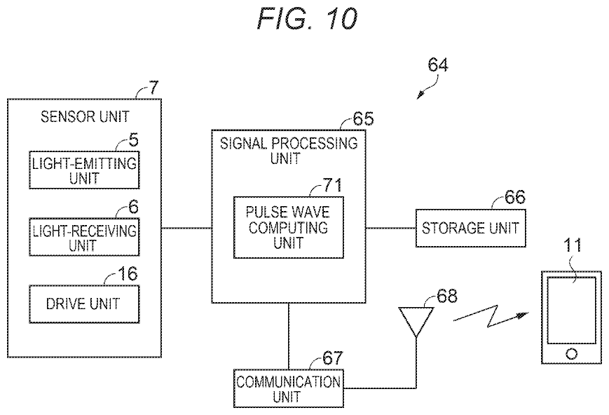

FIG. 10 is a block diagram showing the configuration for electrical control in the biological information measuring device. In FIG. 10, the biological information measuring device 1 has a control unit 64 controlling operations of the biological information measuring device 1. The control unit 64 has a signal processing unit 65 performing various kinds of computational processing, and a storage unit 66 storing various kinds of information. The sensor unit 7 and a communication unit 67 are coupled to the control unit 64.

The communication unit 67 has a modulation circuit and a demodulation circuit for wireless communication. An antenna 68 is coupled to the communication unit 67. The communication unit 67 performs communication processing, for example, for short-range wireless communication such as Bluetooth (trademark registered) with a terminal device such as the smartphone 11. Specifically, the communication unit 67 performs reception processing to receive a signal from the antenna 68 and transmission processing to transmit a signal to the antenna 68. The functions of the communication unit 67 can be implemented by a processor for communication or by a logic circuit such as an ASIC (application-specific integrated circuit). The communication unit 67 wirelessly communicates pulse information such as the number of pulse beats computed by the signal processing unit 65, from the antenna 68 to the smartphone 11.

An operator operates the smartphone 11 to set an operation of the biological information measuring device 1 or give an instruction. The smartphone 11 transmits instruction information to the biological information measuring device 1. The communication unit 67 receives the instruction information from the smartphone 11. Therefore, the smartphone 11 displays the operation instruction to the biological information measuring device 1 and data of the pulse wave and the number of pulse beats detected by the biological information measuring device 1.

The storage unit 66 is formed of a semiconductor memory such as a RAM or ROM. The storage unit 66 stores a program describing an operation control procedure for the biological information measuring device 1 and a pulse wave computation procedure. The storage unit 66 also stores data of the pulse wave signal outputted from the sensor unit 7. The storage unit 66 also has a storage area functioning as a work area, temporary file or the like for the signal processing unit 65 to operate with, and various other storage areas.

The signal processing unit 65 performs various kinds of signal processing and control processing, for example, using the storage unit 66 as a work area. The signal processing unit 65 is implemented, for example, by a processor such as a CPU (central processing unit), or a logic circuit such as an ASIC (application-specific integrated circuit).

The signal processing unit 65 has a pulse wave computing unit 71. The pulse wave computing unit 71 takes in data of a pulse wave signal from the sensor unit 7 and performs computation processing to computer pulse information. The pulse information is, for example, information about the number of pulse beats or the like. Specifically, the pulse wave computing unit 71 performs frequency analysis processing such as FFT (fast Fourier transform) onto the pulse wave signal and thus finds the spectrum of the pulse wave signal. The signal processing unit 65 then increases sixtyfold a frequency with a high intensity in the resulting spectrum and thus calculates the number of pulse beats. However, the pulse information is not limited to the number of pulse beats itself and may be, for example, the frequency or period of the pulse wave, or the like. The pulse information may also include data of change in the number of pulse beats with time.

As described above, this embodiment has the following effects.

(1) According to this embodiment, the biological information measuring device 1 has the passage part 4 and the back lid 3 supporting the passage part 4. The contact surface 3a of the back lid 3 comes into contact with the arm 12. The irradiation light 31 and the reflected light 32 pass through the passage part 4. The outer surface part 4a of the passage part 4 comes into contact with the arm 12. The outer surface part 4a has the convex surface 4b. The contact surface 3a and the convex surface 4b come into contact with the arm 12. The convex surface 4b protrudes from the contact surface 3a and along the first direction 24 and presses the arm 12.

The inner surface part 4c of the passage part 4 has the recess part 4d recessed toward the convex surface 4b. The bottom surface 4e of the recess part 4d is provided between the contact surface 3a and the convex surface 4b as viewed along a cross section taken from the second direction 25. Therefore, the distance between the light-emitting unit 5 and the light-receiving unit 6, and the arm 12, can be made shorter than when the bottom surface 4e of the recess part 4d is located further to the side opposite to the convex surface 4b than the contact surface 3a. When the distance between the light-emitting unit 5 and the light-receiving unit 6, and the arm 12, is shorter, the light-receiving unit 6 can receive the reflected light 32 with a higher intensity than when the distance between the light-emitting unit 5 and the light-receiving unit 6, and the arm 12, is longer. Specifically, the intensity of the reflected light 32 changes in proportion to the square of the distance. Receiving the reflected light 32 with a higher intensity increases the amplitude of the pulse wave signal. The biological information measuring device 1 can detect an intense pulse wave signal, since the distance between the light-emitting unit 5 and the light-receiving unit 6, and the arm 12, can be made shorter.

(2) According to this embodiment, the site where the irradiation light 31 is emitted from the light-emitting unit 5 is arranged nearer to the convex surface 4b than the contact surface 3a. The distance between the light-emitting unit 5 and the arm 12 can be made shorter than when the site where the irradiation light 31 is emitted is arranged further to the side opposite to the convex surface 4b than the contact surface 3a. In the biological information measuring device 1, the distance between the light-emitting unit 5 and the arm 12 can be made shorter and therefore the light-receiving unit 6 can receive the reflected light 32 with a higher intensity. Therefore, the biological information measuring device 1 can detect an intense pulse wave signal.

(3) According to this embodiment, the recess part 4d is arranged in the passage part 4. The sensor unit 7 is arranged in the recess part 4d. In this case, the length from the sensor substrate 14 to the apex 4g of the passage part 4, as viewed along a cross section taken from the second direction 25, can be made shorter. Therefore, the biological information measuring device 1 can be reduced in thickness. Also, since the length from the sensor substrate 14 to the apex 4g of the passage part 4 is short, the light-receiving unit 6 can receive the reflected light 32 with a high intensity even when the light-emitting unit 5 is driven with low electric power. Thus, the biological information measuring device 1 can detect an intense pulse wave signal, with lower electric power.

### Second Embodiment

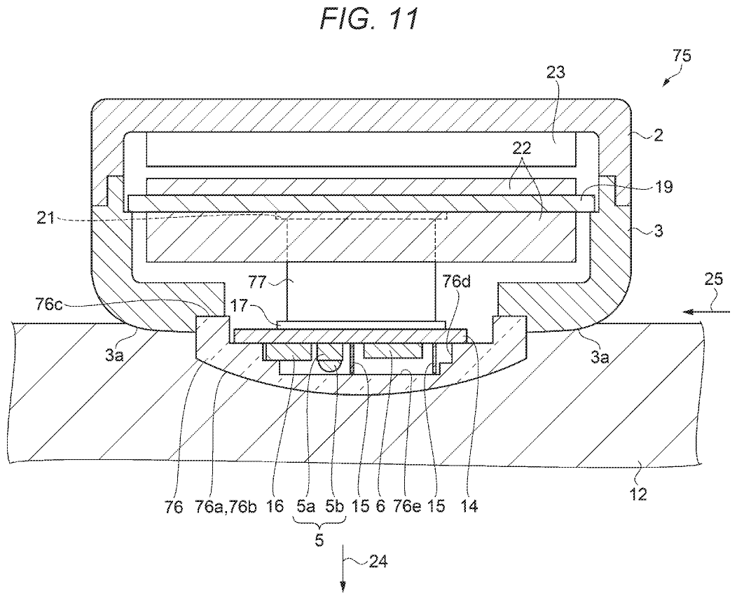

Another embodiment of the biological information measuring device will now be described with reference to the schematic side cross-sectional view of FIG. 11 showing the structure of the biological information measuring device. This embodiment differs from the first embodiment in the shape of the passage part 4 shown in FIG. 4. The same features as in the first embodiment will not be described further in detail.

In this embodiment, a biological information measuring device 75 has a passage part 76 supported by the back lid 3, as shown in FIG. 11. The passage part 76 is light-transmissive. Therefore, the irradiation light 31 emitted from the light-emitting unit 5 passes through the passage part 76. Also, the reflected light 32 reflected off the arm 12 passes through the passage part 76. The first direction 24 is a direction from the light-emitting unit 5 toward the passage part 76. A part of the passage part 76 that faces into the first direction 24 is defined as an outer surface part 76a. The outer surface part 76a comes into contact with the arm 12. The contact surface 3a surrounds the passage part 76. The outer surface part 76a has a convex surface 76b protruding from the contact surface 3a and along the first direction 24 and coming into contact with the arm 12.

A part of the passage part 76 that is in a front-back relationship with the outer surface part 76a is defined as an inner surface part 76c. The inner surface part 76c is a part of the surface of the passage part 76 that faces in the direction opposite to the first direction 24. The inner surface part 76c has a recess part 76d, as viewed along a cross section taken from the second direction 25. The recess part 76d has a bottom surface 76e provided between the contact surface 3a and the convex surface 76b.

A surface of the sensor substrate 14 that faces into the first direction 24 is in contact with the inner surface part 76c. The light-emitting unit 5, the light-receiving unit 6, the light-shielding wall 15, and the drive unit 16 are accommodated in the recess part 76d. The first connector 17 and the second connector 21 are electrically coupled together by a flat cable 77. The entirety of the light-emitting unit 5 protrudes from the contact surface 3a and along the first direction 24. In the first direction 24 from the light-emitting unit 5, the distance between the outer surface part 76a and the bottom surface 76e is short and therefore the light-emitting unit 5 can irradiate the arm 12 with the irradiation light 31 having a high intensity.

The passage part 76 protrudes further from the contact surface 3a into the first direction 24 than the passage part 4 in the first embodiment. In this case, the passage part 76 can press an arteriole more strongly than the passage part 4. Also, the light-emitting unit 5 and the light-receiving unit 6 protrude into the first direction 24. The biological information measuring device 75 can detect an intense pulse wave signal since the distance between the light-emitting unit 5 and the arm 12 can be made shorter.

### Third Embodiment

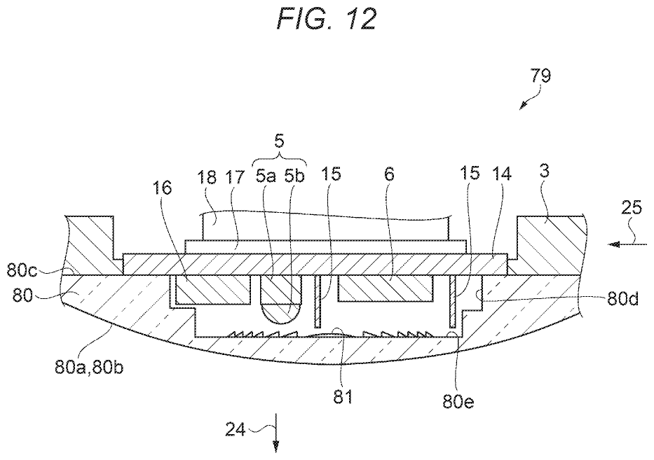

Another embodiment of the biological information measuring device will now be described with reference to the schematic side cross-sectional view of FIG. 12 showing the structure of the passage part. This embodiment differs from the first embodiment in the shape of the passage part shown in FIG. 5. The same features as in the first embodiment will not be described further in detail.

In this embodiment, a biological information measuring device 79 has a passage part 80 supported by the back lid 3, as shown in FIG. 12. The passage part 80 is light-transmissive. Therefore, the irradiation light 31 emitted from the light-emitting unit 5 passes through the passage part 80. Also, the reflected light 32 reflected off the arm 12 passes through the passage part 80.

A part of the passage part 80 that faces into the first direction 24 is defined as an outer surface part 80a. The outer surface part 80a comes into contact with the arm 12. The contact surface 3a surrounds the passage part 80. The outer surface part 80a has a convex surface 80b protruding from the contact surface 3a and along the first direction 24 and coming into contact with the arm 12. A part of the passage part 80 that is in a front-back relationship with the outer surface part 80a is defined as an inner surface part 80c. The inner surface part 80c has a recess part 80d, as viewed along a cross section taken from the second direction 25. The recess part 80d has a bottom surface 80e provided between the contact surface 3a and the convex surface 80b.

The bottom surface 80e has a lens 81. The lens 81 is not particularly limited, provided that it is a convex lens. In this embodiment, for example, a Fresnel lens is arranged as the lens 81. The Fresnel lens can be thin in the first direction 24 and therefore enables the light-emitting unit 5 to be close to the convex surface 80b.

Adjusting the focal length of the lens 81 enables the lens 81 to adjust the site where the irradiation light 31 and the reflected light 32 passing through the passage part 80 are condensed. This can increase the proportion at which the reflected light 32 passing through the passage part 80 irradiates the light-receiving unit 6. Thus, the biological information measuring device 79 can pressurize the arm 12 and detect a pulse wave signal with a high sensitivity.

### Fourth Embodiment

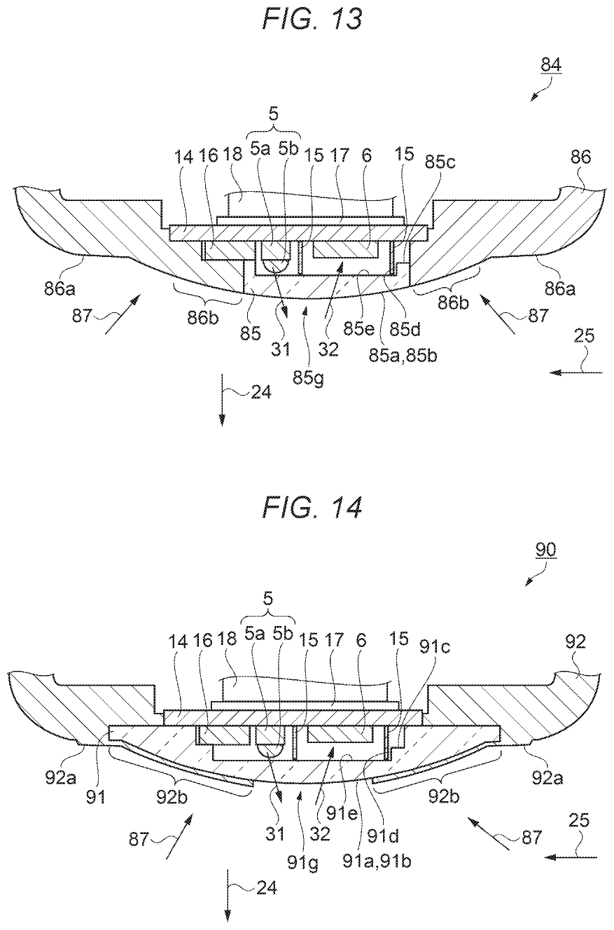

Another embodiment of the biological information measuring device will now be described with reference to the schematic side cross-sectional view of FIG. 13 showing the structure of the back lid. This embodiment differs from the first embodiment in the shape of the back lid 3 shown in FIG. 4. The same features as in the first embodiment will not be described further in detail.

In this embodiment, a biological information measuring device 84 has a passage part 85 supported by a back lid 86, as shown in FIG. 13. The passage part 85 transmits light. The back lid 86 including a light-shielding part 86b does not transmit light 87. The first direction 24 is a direction from the light-emitting unit 5 toward the passage part 85. A contact surface 86a coming into contact with the arm 12 is arranged on a side of the back lid 86 that faces into the first direction 24. On the surface of the back lid 86 that faces into the first direction 24, the contact surface 86a is a planar part. A part of the passage part 85 that faces into the first direction 24 is defined as an outer surface part 85a. The outer surface part 85a comes into contact with the arm 12. The outer surface part 85a has a convex surface 85b protruding from the contact surface 86a and along the first direction 24 and coming into contact with the arm 12.

A part of the passage part 85 that is in a front-back relationship with the outer surface part 85a is defined as an inner surface part 85c. The inner surface part 85c has a recess part 85d, as viewed along a cross section taken from the second direction 25. The recess part 85d has a bottom surface 85e provided between the contact surface 86a and the convex surface 85b.

The back lid 86 has the light-shielding part 86b, which does not pass the light 87. The light-shielding part 86b is located around the passage part 85. The light-shielding part 86b is provided between the contact surface 86a and an apex 85g of the convex surface 85b, as viewed along a cross section taken from the second direction 25.

The light-shielding part 86b does not pass the light 87. Therefore, the light-shielding part 86b restrains the light-receiving unit 6 from receiving the light 87 other than the reflected light 32 passing through the passage part 85. The light 87 other than the reflected light 32 passing through the passage part 85 is not involved in the pulsation of the blood vessel. The light 87 becomes a noise component when received by the light-receiving unit 6. The light-shielding part 86b restrains the light-receiving unit 6 from receiving the light 87, which becomes a noise component. Therefore, the biological information measuring device 84 can detect a pulse wave signal with a high accuracy.

### Fifth Embodiment

Another embodiment of the biological information measuring device will now be described with reference to the schematic side cross-sectional view of FIG. 14 showing the structure of the back lid. This embodiment differs from the first embodiment in the shape of the back lid 3 shown in FIG. 4. The same features as in the first embodiment will not be described further in detail.

In this embodiment, a biological information measuring device 90 has a passage part 91 supported by a back lid 92, as shown in FIG. 14. The passage part 91 transmits light. The back lid 92 including a light-shielding part 92b does not transmit the light 87. The first direction 24 is a direction from the light-emitting unit 5 toward the passage part 91. A contact surface 92a coming into contact with the arm 12 is arranged on a side of the back lid 92 that faces into the first direction 24. On the surface of the back lid 92 that faces into the first direction 24, the contact surface 92a is a planar part. A part of the passage part 91 that faces into the first direction 24 is defined as an outer surface part 91a. The outer surface part 91a comes into contact with the arm 12. The outer surface part 91a has a convex surface 91b protruding from the contact surface 92a and along the first direction 24 and coming into contact with the arm 12.

A part of the passage part 91 that is in a front-back relationship with the outer surface part 91a is defined as an inner surface part 91c. The inner surface part 91b is a part of the surface of the passage part 91 that faces in the direction opposite to the first direction 24. The inner surface part 91c has a recess part 91d, as viewed along a cross section taken from the second direction 25. The recess part 91d has a bottom surface 91e provided between the contact surface 92a and the convex surface 91b.

The back lid 92 has the light-shielding part 92b in the shape of a film, which does not pass the light 87. The light-shielding part 92b covers the side of the passage part 91 that faces in the first direction 24, except for the side of the light-emitting unit 5 and the light-receiving unit 6 that faces in the first direction 24. The light-shielding part 92b is provided between the contact surface 92a and an apex 91g of the convex surface 91b, as viewed along a cross section taken from the second direction 25.

The light-shielding part 92b does not pass the light 87. Therefore, the light-shielding part 92b restrains the light-receiving unit 6 from receiving the light 87 other than the reflected light 32 passing through the passage part 91. The light 87 other than the reflected light 32 passing through the passage part 91 is not involved in the pulsation of the blood vessel. The light 87 becomes a noise component when received by the light-receiving unit 6. The light-shielding part 92b restrains the light-receiving unit 6 from receiving the light 87, which becomes a noise component. Therefore, the biological information measuring device 90 can detect a pulse wave signal with a high accuracy.

The present disclosure is not limited to the foregoing embodiments. A person having ordinary skills in the art can add various changes and improvements within the range of the technical idea according to the present disclosure. Modification examples are given below.

### Modification Example 1

In the first embodiment, the biological information measuring device 1 does not have a display unit, and the smartphone 11 displays pulse information. However, the biological information measuring device 1 may have a display unit. The pulse information may be displayed on the display unit of the biological information measuring device 1. The operator can check the pulse information even when not having the smartphone 11. The biological information measuring device 1 may also have an instruction unit such as an operation button. The operator can give an instruction to start and stop measuring or the like even when not having the smartphone 11.

### Modification Example 2

In the first embodiment, the biological information measuring device 1 wirelessly communicates with the smartphone 11. However, the biological information measuring device 1 has the external connector 13 and therefore may perform wired communication with an external device via the external connector 13. The biological information measuring device 1 can communicate with a device that does not have a wireless communication function.

### Modification Example 3

In the first embodiment, the biological information measuring device 1 is mounted on the arm 12. However, the place to mount the biological information measuring device 1 is not limited the arm 12 and may be a leg or foot. In this case, the length of the first band 8 and the second band 9 can be adjusted to the leg or foot to arrange the biological information measuring device 1 on the leg or foot.

The biological information measuring device 1 may also be fixed to the abdomen or back with an adhesive tape. In this case, the first band 8 and the second band 9 may be removed from the biological information measuring device 1. This enables the biological information measuring device 1 to be fixed more easily with an adhesive tape. When arranged on the skin where an arteriole exists, the biological information measuring device 1 can detect the pulsation of the blood vessel 53. Therefore, when the biological information measuring device 1 cannot be arranged on the arm 12 because of a plaster cast mounted on the arm 12 or for similar reasons, the biological information measuring device 1 can be arranged at another site of the human body. The biological information measuring device 1 may also be mounted on other animals than human. In this case, the biological information measuring device 1 can similarly measure the number of pulse beats of the animals.

### Modification Example 4

In the fourth embodiment, the light-shielding part 86b is arranged in the back lid 86. The light-shielding part 86b may be provided in the passage part. In the passage part 4 in the first embodiment, the side facing in the first direction 24 of the light-emitting unit 5 and the light-receiving unit 6 is made light-transmissive. The other parts of the passage part 4 than the side facing in the first direction 24 of the light-emitting unit 5 and the light-receiving unit 6 may be formed of a material that does not transmit the light 87. In this case, the light-receiving unit 6 can be similarly restrained from receiving the light 87 other than the reflected light 32.

### Modification Example 5

In the fifth embodiment, the light-shielding part 92b is arranged in the back lid 92. The light-shielding part 92b may be provided in the passage part. In the passage part 4 in the first embodiment, a film that does not transmit the light 87 may be arranged at the parts other than the side facing in the first direction 24 of the light-emitting unit 5 and the light-receiving unit 6. In this case, the light-receiving unit 6 can be similarly restrained from receiving the light 87 other than the reflected light 32.

In the modification example 5 and the fifth embodiment, when the light-shielding part 92b has a large thickness, the pressure may be released by the step between the light-shielding part 92b and the outer surface part 91a, thus making it difficult to apply an appropriate pressure. Therefore, a light-transmissive film having a thickness equivalent to that of the light-shielding part 92b may be provided at the apex 91g. Alternatively, the apex 91g may protrude into the first direction 24.

The contents derived from the embodiments will now be described.

A biological information measuring device includes: a light-emitting unit emitting irradiation light irradiating a living body; a light-receiving unit receiving reflected light of the irradiation light reflected off the living body; a passage part where the irradiation light and the reflected light pass; and a back lid having a contact surface which surrounds the passage part and comes into contact with the living body, the back lid supporting the passage part. The passage part has an outer surface part coming into contact with the living body, and an inner surface part in a front-back relationship with the outer surface part. The outer surface part has a convex surface protruding from the contact surface and along a first direction from the light-emitting unit toward the passage part and coming into contact with the living body. The inner surface part has a recess part having a bottom surface provided between the contact surface and the convex surface as viewed along a cross section taken from a second direction orthogonal to the first direction.

According to this configuration, the light-emitting unit emits irradiation light to a living body, and the light-receiving unit receives a part of reflected light reflected off the living body. The blood vessel in the living body absorbs a part of the irradiation light. Since the blood forms a pulsatile flow in the blood vessel, the reflected light changes with time with an intensity reflecting the pulsatile flow. The biological information measuring device measures the reflected light and thus detects the pulsation of the blood vessel.

The biological information measuring device has the passage part and the back lid supporting the passage part. In the back lid, the contact surface comes into contact with the living body. The irradiation light and the reflected light pass through the passage part. The site coming into contact with the living body, of the passage part, is the outer surface part. The outer surface part has the convex surface. The contact surface and the convex surface come into contact with the living body. The direction from the light-emitting unit toward the passage part is the first direction. The direction orthogonal to the first direction is the second direction. The convex surface protrudes from the contact surface and along the first direction and presses the living body.

The site in a front-back relationship with the outer surface part, of the passage part, is the inner surface part. The inner surface part has the recess part recessed toward the convex surface. The bottom surface of the recess part is provided between the contact surface and the convex surface as viewed along a cross section taken from the second direction. The distance between the light-emitting unit and the light-receiving unit, and the living body, can be made shorter than when the bottom surface is located further into the direction opposite to the first direction than the contact surface. A signal obtained by measuring the pulsation of the blood vessel is a pulse wave signal. When the distance between the light-emitting unit and the light-receiving unit, and the living body, is shorter, a stronger pulse wave signal is detected than when the distance between the light-emitting unit and the light-receiving unit, and the living body, is longer. This biological information measuring device can detect a stronger pulse wave signal because the distance between the light-emitting unit and the light-receiving unit, and the living body, can be made shorter.

In the biological information measuring device, at least a part of the light-emitting unit may protrude from the contact surface and along the first direction.

According to this configuration, the site where the light-emitting unit emits the irradiation light is located nearer to the convex surface than the contact surface. The distance between the light-emitting unit and the living body can be made shorter than when the site where the irradiation light is emitted is located further to the side opposite to the convex surface than the contact surface. This biological information measuring device can detect a stronger pulse wave signal because the distance between the light-emitting unit and the living body can be made shorter.

In the biological information measuring device, an apex of the convex surface may coincide with a middle point of a straight line connecting a center of the light-emitting unit and a center of the light-receiving unit, as viewed in a plan view taken from the first direction.

According to this configuration, the apex of the convex surface coincides with the middle point of the straight line connecting the center of the light-emitting unit and the center of the light-receiving unit. The apex of the convex surface powerfully pressurizes the living body. Therefore, the vicinity of the apex of the convex surface is a site where the pulsation of the blood vessel changes largely. When viewed from the first direction, the site where the pulsation of the blood vessel changes largely is the middle point of the straight line connecting the center of the light-emitting unit and the center of the light-receiving unit. The middle point of the straight line connecting the center of the light-emitting unit and the center of the light-receiving unit is defined as an intermediate point. An inner part of the living body located in the first direction at the intermediate point is defined as a part to be measured.

The part to be measured is a site where the biological information measuring device can measure change in the pulsation of the blood vessel with a high sensitivity. As the apex of the convex surface pressurizes the part to be measured, the biological information measuring device can measure a site where the pulsation of the blood vessel changes largely, with a high sensitivity. Even when the biological information measuring device moves along the surface of the living body during exercise or the like, the biological information measuring device measures the site where the pulsation of the blood vessel changes largely, with a high sensitivity. Therefore, the biological information measuring device can stably measure the pulsation of the blood vessel.

In the biological information measuring device, the bottom surface may have a lens.

According to this configuration, in the biological information measuring device, the lens is provided at the bottom surface of the recess part. Adjusting the focal length of the lens enables adjustment of the site where the light and passing through the passage part is condensed. This can increase the proportion at which the light passing through the passage part irradiates the light-receiving unit. Thus, this biological information measuring device can detect a pulse wave signal with a high sensitivity.

In the biological information measuring device, the back lid may have a light-shielding part where light does not pass. The light-shielding part may be provided between the contact surface and an apex of the convex surface, as viewed along a cross section taken from the second direction.

According to this configuration, the light-shielding part is provided between the contact surface and the apex of the convex surface. The light-shielding part does not pass light. Therefore, the light-shielding part restrains the light-receiving unit from receiving the light other than the reflected light passing through the passage part. The light other than the reflected light passing through the passage part is not involved in the pulsation of the blood vessel and therefore becomes a noise component when received by the light-receiving unit. The light-shielding part restrains the light-receiving unit from receiving the light which becomes a noise component. Therefore, the biological information measuring device can detect a pulse wave signal with a high accuracy.

What is claimed is:

1. A biological information measuring device comprising:
a light-emitting unit emitting irradiation light irradiating a living body;
a light-receiving unit receiving reflected light of the irradiation light reflected off the living body;
a passage part where the irradiation light and the reflected light pass; and
a back lid having a contact surface which surrounds the passage part and comes into contact with the living body, the back lid supporting the passage part, wherein
the passage part has
an outer surface part coming into contact with the living body, and
an inner surface part in a front-back relationship with the outer surface part,
the outer surface part has a convex surface protruding from the contact surface and along a first direction from the light-emitting unit toward the passage part and coming into contact with the living body, and
the inner surface part has a recess part having a bottom surface provided between the contact surface and the convex surface as viewed along a cross section taken from a second direction orthogonal to the first direction.

2. The biological information measuring device according to claim 1, wherein
at least a part of the light-emitting unit protrudes from the contact surface and along the first direction.

3. The biological information measuring device according to claim 1, wherein
an apex of the convex surface coincides with a middle point of a straight line connecting a center of the light-emitting unit and a center of the light-receiving unit, as viewed in a plan view taken from the first direction.

4. The biological information measuring device according to claim 1, wherein
the bottom surface has a lens.

5. The biological information measuring device according to claim 1, wherein
the back lid has a light-shielding part where light does not pass, and the light-shielding part is provided between the contact surface and an apex of the convex surface, as viewed along a cross section taken from the second direction.
